(19) United States
(12) Patent Application Publication
(10) Pub. No.: US 2004/0044328 A1
(43) Pub. Date: Mar. 4, 2004

(54) Applicator and stimulator device

(76) Inventors: Donald Jack Kemp, Chichester (GB); Elizabeth Paul, Harrogate (GB)

Correspondence Address: NIXON & VANDERHYE, PC, 1100 N GLEBE ROAD, 8TH FLOOR, ARLINGTON, VA, 22201-4714, US

(21) Appl. No.: 10/416,833

(22) PCT Filed: Nov. 12, 2001
(86) PCT No.: PCT/GB01/04999

(30) Foreign Application Priority Data
Nov. 17, 2000 (GB) 0028019.8
Oct. 1, 2001 (GB) 0123523.3

(51) Int. Cl. A61M 31/00

(52) U.S. Cl. 604/500

(57) ABSTRACT

An applicator and stimulator device comprises a flexible sleeve closed at one end and having a one or more indented cells in its outer wall and adapted to accomodate a human finger within the sleeve. In order to assist application of fluids it may have one or more cells associated with the outer wall which contain fluid which is retained by a seal. The seal is openable or rupturable when pressure is applied across the wall or the wall is in contact with an environment that weakens the seal. The sleeve is formed from an elastic material such as natural or a synthetic rubber such as a highly plasticised PVC resin. The cells associated with the outer wall are formed by creating depressions in the wall during moulding of the device. In its unflexed state the body of the sleeve is substantially cylindrical and the cells are preferably formed on a portion of the sleeve surface near to the closed end sustaining and angle of about 90 degrees over the surface measured from the axis of the cylinder. The cells may contain a lubricant, a pharmacologically active compound or a combination of such substances. The preferred pharmacologically active compound is a vasoactive compound such as vasodilator alone or in admixture with one or more adjuvants to ensure consistency and retention when applied to tissue. At least part of the periphery of the open end of the sleeve may carry a sealed channel filled with a lubricant that is released when the seal is ruptured.

This invention relates to a stimulator and a method and an applicator device for the topical application, by digital means, of fluids to sensitive skin areas and particularly erectile tissue.

The digital massaging of sensitive skin areas is preferably performed using a finger contained within a flexible sleeve. This has the dual advantages of preventing infection and eliminating the wide differences between the skin surface of different masseurs. Use of a sleeve with totally smooth latex rubber surface, such as those commonly available as “finger stalls”, seldom provides sufficient stimulation for erotic purposes.

Direct digital application of fluids, such as lubricants and medicaments, to sensitive skin areas is conventionally carried out by dipping a finger, frequently enclosed in a latex or similar glove, into a container of the fluid and thereafter placing the finger on the area to be treated. To achieve appreciable transfer of fluid it is necessary for it to be in a viscous or a thixotropic form such as a cream or paste. The transfer process is inefficient and the need to apply the fluid in cream or paste form requires additives, such as gelling agents, which can lower the concentration of any active ingredient, such as a medicament, present in the fluid. The need to carry both a container and an applicator glove can on occasions be extremely inconvenient. This is particularly so when the fluid is a lubricant and/or medicament that is to be applied to the clitoral area of a female prior to congress.

The present invention provides an improved stimulator device for digital stimulation of the clitoris, a method and device for the topical application, by digital means, of fluids such as lubricants and medicaments to sensitive skin areas such as erectile tissue.

According to the present invention there is provided a stimulator device comprising a flexible sleeve closed at one end and having a one or more indented cells in its outer wall and adapted to accommodate a human finger.

There is further provided an applicator device comprising a flexible sleeve closed at one end and including one or more cells associated with its outer wall which contain fluid which is retained by a seal which is openable or rupturable when pressure is applied across the wall.

There is also provided a method of applying fluids to skin areas characterised in that an applicator device comprising a flexible sleeve closed at one end and including one or more sealed cells associated with its outer wall containing a fluid is fitted over a finger, and thereafter the area of the outer wall adjacent the cells is placed in contact with the skin area using gentle pressure whereby the fluid is released from the cell or cells through rupture or opening of the seal.

The flexible sleeve is formed from an elastic material such as natural or a synthetic rubber. The sleeve may be formed by dipping a mould into a latex, injection moulding, blow moulding or other fabrication method; such methods are well known. The cells associated with the outer wall may be formed by creating depressions in the wall either during moulding of the applicator or subsequently. In its unflexed state the body of the sleeve will be substantially cylindrical. The cells are preferably formed on a portion of the sleeve surface near to the closed end and sustaining an angle of about 90 degrees over the surface measured from the axis of the cylinder. A preferred material for moulding the applicator sleeves is highly plasticised polyvinyl chloride (PVC) resin.

In one embodiment the cells are open ended and covered with a layer of sealant after filling with fluid. In an alternative embodiment the cells have mouths which are substantially self-sealed through the resilience of the material from which they are formed. In a further embodiment the cells are formed separately from the sleeve and are attached by an adhesive or thermal welding to the outer surface of the sleeve. The sealing of the cells after placing fluid in them may be carried out by adhering a thin film over the area of the sleeve containing the cells. The film must be sufficiently thin that it can be ruptured by pressure across the sleeve wall. In preferred embodiments the film is formed from material which is water dispersible so that a moist environment assists rupture by weakening the film. The film may also be pH sensitive so that the rupture may be assisted by a change in pH caused by contact with body fluids. In an alternative embodiment the sealing of the cells after placing fluid in them may be carried out by coating a thin layer of sealant or varnish over the area of the sleeve containing the cells. Suitable film forming materials for use as films or as sealant coatings include gelatin or modified gelatins, polyvinyl alcohol, cellulose or modified cellulose such as carboxy methyl cellulose, polyvinylpyrrolidone, polyvinyl acetate and alginic acid derivatives.

The fluid content of the cells may comprise a lubricant, a pharmacologically active compound or a combination of such substances. When the fluid is to be released on or around a human clitoris the pharmacologically active compound may be a vasoactive compound such as a vasodilator, a counter irritant, a nerve stimulant, and/or a lubricant. Among the pharmacologically active compounds that may be contained in the cells are glyceryl trinitrate, isosorbide dinitrate, aminophylline, theophylline with ethylene diamine, co-dergocrine messylate, a combination of dihydroergogornime messylate and dihydroergocristinne messylate with alpha and beta dihydroergocrytine messylate, prostanoid compounds such as prostaglandin-E, apomorphine, menthol alone or with L-alanine. The fluid into. each cell either successively or simultaneously. The dispensing system may be automated to provide rapid filling of the cells in each sleeve. After filling a layer of varnish composition may contain adjuvants to ensure consistency and retention when applied to tissue such as stearic acid, tri-ethanolamine, silicone oils, cetyl alcohol, glycerol, acidic carboxyvinyl gelling agents, methyl cellulose, wax emulsifiers and preservatives such as antibacterial and antifungal agents When the fluid cells are provided within the wall of the applicator sleeve they may be filled by dispensing controlled volumes of fluid after which a sealant coating or a thin film is applied to ensure that the fluid is retained in each of the cells. In an alternative embodiment in which the cells are attached to the surface of the sleeve subsequent to its manufacture the cells are created and filled with fluid by known processes such as coaservation whereby a suspension of substantially spherical cells are produced suspended in a liquid medium. The suspension may be applied directly to the sleeve or the cells may be first separated. In either case the applied composition will be of a nature or contain additives to ensure adhesion to the sleeve surface.

The sleeve, particularly when it is used as a stimulator device with unfilled cells, may carry a sealed channel around part of the periphery of the open end. The channel is filled with an aqueous or non-aqueous lubricant that is released when the seal is ruptured. Rupture will occur when the base of the sleeve is pressed against vulval tissue. The sealants used for the channel can be the same as those described for sealing the fluid containing cells on the sleeve wall.

In order that the invention may be clearly understood one form thereof will now be described with reference to the accompanying drawing, in which:

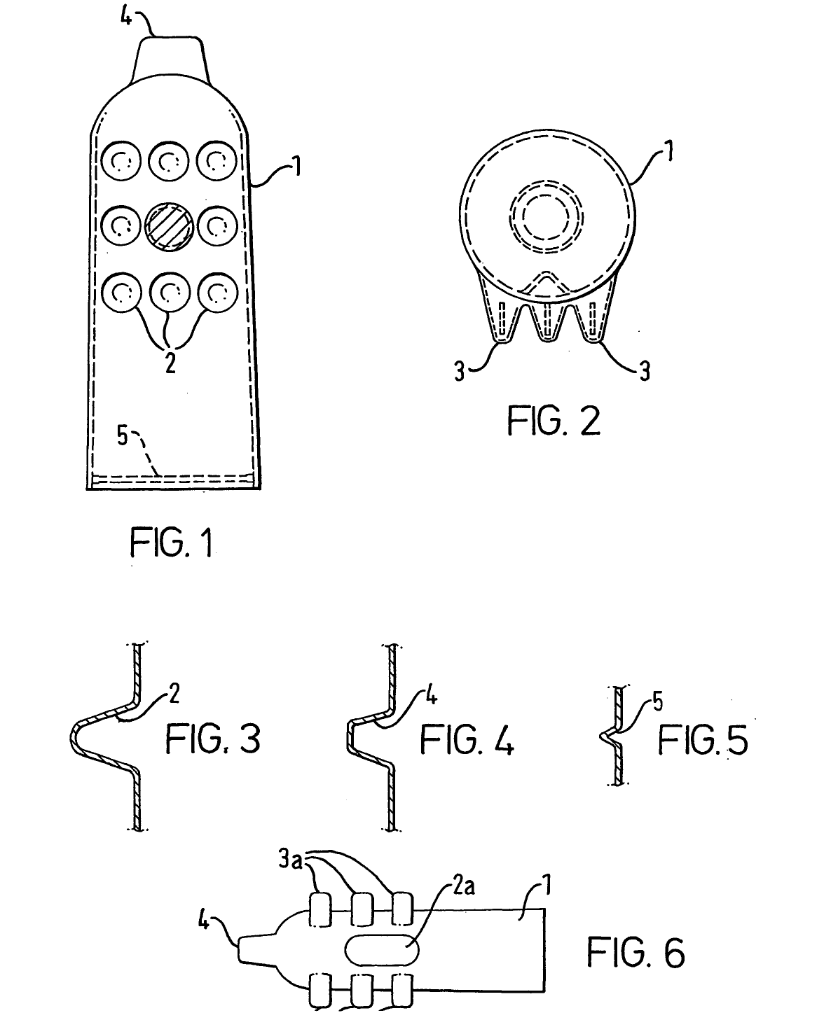

FIG. 1 is a plan view above the major axis of a stimulator or applicator sleeve in accordance with the invention,

FIG. 2 is a cross sectional view across the sleeve shown in FIG. 1,

FIG. 3 a cross sectional view of a cell within the wall of the sleeve shown in FIG. 1,

FIG. 4 is a cross sectional view of the terminal projection at the closed end of the sleeve shown in FIG. 1,

FIG. 5 is a cross sectional view of a channel at the open end of the sleeve shown in FIG. 1, and

FIG. 6 is a plan view above the major axis of an alternative embodiment of a stimulator or applicator sleeve in accordance with the invention.

A stimulator or applicator device according to the invention, see FIG. 1, consists of a sleeve fabricated from a flexible and resilient material having a wall 1 carrying a series of indentations 2. The indentations 2, see FIG. 3, are open at their outwardly facing ends when the device is to be used as a stimulator. Alternatively the indentations 2 must be closed at their outwardly facing ends when the device is to be used as a fluid applicator. The sealed indentations can act as fluid containing cells. When filled with fluid the open ends of the indentations or cells are sealed with a thin fluid-tight layer formed by the application of a sealant varnish layer or a thin adhesive film. The varnish or film must be inert and insoluble with regard to the contained fluid. When a finger is placed within the sleeve digital pressure on to a surface will cause the varnish layer or film to rupture, the cell to collapse and the contained fluid to flow on to the surface. In the case of some varnish or film layers rupture may be assisted by the change of pH, temperature end/or the presence of moisture when the sleeve is inserted into body cavities.

Optionally the wall proximate to the open end of the sleeve may carry a projections 3 which can act as a guide and/or a tactile stimulant. The closed end of the sleeve may also carry a projection 4 for this purpose.

In the stimulator embodiment the sleeve wall 1 carries a channel 5 around part of the periphery of the open end. The channel 5 is preferably filled with a lubricant fluid and is sealed in a manner similar to that used for the indentations 2.

In an example an applicator sleeve was constructed of soft PVC having a length of approximately 65 mm and an internal diameter of about 17 mm. The outer surface carried a series of cells formed as indentations 7 mm deep having a frustro-conical shape and a surface diameter of 5 mm. The cells were formed as three rows of three in a pattern on a portion of the wall. A finger inserted into the sleeve was able to rupture some or all of the cells when pressure was applied to an adjacent surface but without such pressure the cells remained intact retaining their fluid content.

In an alternative embodiment, see FIG. 6, the sleeve 1 has projections 3a which can act as a guide and/or a tactile stimulant on the upper surface of the sleeve. Between the projections 3a lies an indentation 2a. After filling with fluid the open end of the indentation 2a is sealed with a thin fluid-tight layer as described above.

The applicator device is particularly useful in the treatment of female sexual arousal disorder (FSAD). For this purposed the cells of the applicator contain a fluid composition which includes a substance having vasodilatory properties. When the applicator is pressed against the clitoris the vasodilator fluid is released and the effect is dilation of the local arterial blood supply vessels which causes clitoral engorgement and automatic vaginal lubrication.

1. A stimulator and/or applicator device for vaginal and/or clitoral use, the device comprising a flexible finger sleeve which is closed at one end and adapted to accommodate a human finger, the sleeve having one or more fluid cells defined by indentations in its outer wall and containing a fluid which is suitable for topical application to the clitoral area, each of which fluid cells comprises a seal which is openable or rupturable, so as to release fluid from the cell, when pressure is applied across the sleeve wall or when the wall is in contact with an environment that weakens the seal.

2. A stimulator and/or applicator device as claimed in claim 1, wherein the finger sleeve wall proximate to the closed end of the finger sleeve carries projections which can act as a guide and/or as a tactile stimulant.

3. A stimulator and/or applicator device as claimed in claim 1 or claim 2, characterised in that the flexible finger sleeve is formed from an elastic material such as a natural or synthetic rubber.

4. A stimulator and/or applicator device as claimed in claim 3, characterised in that the elastic material is a highly plasticised polyvinyl chloride (PVC) resin.

5. A stimulator and/or applicator device as claimed in any one of the preceding claims, characterised in that the fluid cells are formed by creating depressions in the wall during its moulding.

6. A stimulator and/or applicator device as claimed in any one of the preceding claims, characterised in that in its unflexed state the body of the finger sleeve is substantially cylindrical.

7. A stimulator and/or applicator device as claimed in any one of the preceding claims, characterised in that the fluid cells are formed on a portion of the sleeve surface near to the closed end and sustaining an angle of about 90 degrees over the surface measured from the axis of the cylinder.

8. A stimulator and/or applicator device as claimed in any one of the preceding claims, characterised in that the fluid cells are open ended and covered with a layer of sealant after filling with fluid.

9. A stimulator and/or applicator device as claimed in claim 8, characterised in that the sealant is a film formed from gelatin, a modified gelatin, polyvinyl alcohol, cellulose, a modified cellulose, polyvinyl pyrrolidone, polyvinyl acetate and/or an alginic acid derivative.

10. A stimulator and/or applicator device as claimed in any one of the preceding claims, characterised in that the fluid cells have mouths which are substantially self-sealed through the resilience of the material from which they are formed.

11. A stimulator and/or applicator device as claimed in any one of the preceding claims, characterised in that the fluid cells contain a lubricant, a pharmacologically active compound or a combination of such substances.

12. A stimulator and/or applicator device as claimed in claim 11, characterised in that the pharmacologically active compound is a vasoactive compound, a counter irritant, a nerve stimulant or a combination of such substances.

13. A stimulator and/or applicator device as claimed in claim 12, characterised in that the fluid cells contain a vasoactive compound such as a vasodilator.

14. A stimulator and/or applicator device as claimed in claim 13, characterised in that the fluid cells contain glyceryl trinitrate, isosorbide dinitrate, aminophylline, theophylline with ethylene diamine, co dergocrine mesylate or a combination of dihydroergogornime mesylate and dihydroergocristinne mesylate with alpha- and beta-dihydroergocrytine mesylate.

15. A stimulator and/or applicator device as claimed in claim 12, characterised in that the fluid cells contain a prostanoid compound such as prostaglandin-E.

16. A stimulator and/or applicator device as claimed in claim 12, characterised in that the fluid cells contain apomorphine or menthol either alone or with L-alanine.

17. A stimulator and/or applicator device as claimed in any one of the preceding claims, characterised in that the fluid in the fluid cells contains one or more adjuvants, such as stearic acid or triethanolamine, to ensure consistency and retention when applied to tissue.

18. A stimulator and/or applicator device as claimed in any one of the preceding claims, characterised in that at least part of the periphery of the open end carries a sealed channel filled with an aqueous or non-aqueous lubricant that is released when the seal is ruptured.

19. A method of applying a fluid to a skin area, characterised in that an applicator device according to any one of the preceding claims is fitted over a finger, and thereafter the area of the outer sleeve wall adjacent the fluid cell(s) is placed in contact with the skin area using gentle pressure whereby the fluid is released from the cell(s) through rupture or opening of the seal(s).
